(19) United States
(12) Patent Application Publication
(10) Pub. No.: US 2007/0221215 A1
(43) Pub. Date: Sep. 27, 2007

(54) EJECTION LIQUID AND METHOD FOR EJECTING THE SAME

(75) Inventors: Masaru Sugita, Tokyo (JP); Naoka Sakurada, Yokohama-shi (JP); Yohei Masada, Tokyo (JP); Hideki Kaneko, Yokohama-shi (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/682,038

(22) Filed: Mar. 5, 2007

(30) Foreign Application Priority Data
Mar. 24, 2006 (JP) 2006-083399

(51) Int. Cl. A61M 15/00; A61M 16/10

(52) U.S. Cl. 128/203.12

(57) ABSTRACT

It is an object of the present invention to provide an ejection liquid (liquid composition) which can be stably ejected as a liquid containing an effective ingredient for treating respiratory disease from an ejection opening provided to an ejection device. A specific surface-active compound is added to an ejection liquid containing an effective ingredient for treating respiratory disease. An ejection liquid which is used for stably ejecting a solution containing an active ingredient for treating respiratory disease according to the principle of an ink jet system, a method which is suitable for ejecting this ejection liquid, and an ejection device are provided. The ejection liquid includes an active ingredient for treating j respiratory disease, a specific surface-active compound, and a liquid medium and is ejected from an ejection opening by an ink jet system.

### BACKGROUND OF THE INVENTION

1. Field of the Invention

The present invention relates to an ejection liquid (liquid composition) which is suitably formed into droplets of a liquid containing at least one active ingredient for treating respiratory disease and relates to a method for ejecting the liquid.

2. Description of the Related Art

Some methods for administering drugs to respiratory organs by inhalation have been conventionally known. The outlines and problems of these methods will now be described.

In a metered dose inhaler (MDI) of aerosol suspension, a propellant is a liquefied nonflammable or noncombustible gas and is used for a single shot. A metered dose spray can be achieved by regulating a unit volume of the liquefied gas. However, there are some problems in the control of droplet size under the above-mentioned conditions. In addition, a propellant may cause an adverse health effect.

Further, in a spray system used for spraying a liquid agent using water or ethanol as a medium, the liquid agent is converted into minute droplets by ejecting the liquid agent with a compressed carrier gas through a capillary. Therefore, theoretically, the spray volume can be controlled by regulating the volume of the liquid agent supplied to the capillary channel. However, the control of droplet size is difficult. In particular, in a spray system, the compressed gas used in the process for converting a liquid agent into minute droplets is also used for forming a gas flow for carrying the sprayed minute droplets. Therefore, it is structurally difficult to change the volume (density) of the minute droplets suspended in the carrier gas flow according to purpose.

In addition, as a method for forming droplets with a narrow size distribution, extremely minute droplets produced by a droplet generator based on a principle of ejecting liquid used in ink jet printing, namely, by utilizing an ink jet system, have been disclosed (refer to U.S. Pat. No. 5,894,841 and Japanese Patent Application Laid-Open No. 2002-248171). Here, the term “ink jet system” means a liquid ejection system in which a liquid to be ejected is introduced into a small chamber and droplets are ejected from an orifice by applying an expelling force to the liquid. As methods for expelling a liquid, the following two are known, for example.

(1) “Thermal ink jet system”: air bubbles for ejecting droplets through an orifice (ejection opening) provided to a chamber are generated by using an electrothermal transducer such as a thin-film resistor.

(2) “Piezo ink jet system”: a liquid is directly expelled through an orifice provided to a chamber by using a piezo oscillator (piezoelectric element).

The chamber and orifice are integrated into a head element. This head element is connected to a liquid source and to a controller for controlling the ejection of droplets.

In order to allow a drug depositing to lung, the drug must have a particle size required for deposition depending on the region of lung. Therefore, it is necessary to control particle sizes of droplets according to the respective regions of the lung. The formation of droplets based on the principle of an ink jet system which can control the droplet size is very favorable.

The above-mentioned desirable particle size suitable for pulmonary inhalation is specifically 1 to 5 μm which is very small compared to the liquid droplet size of the present printer ink for a commercially available printer. Consequently, high surface energy or shear force is applied to a drug solution in a process of ejecting. Therefore, in some cases, it is very difficult to ejection minute droplets suitable for pulmonary inhalation.

The above is a problem accompanied by the formation of droplets based on the principle of the ink jet system. Further, the diameter of an ejection opening for generating droplets suitable for spraying a drug is significantly small compared to that in a known ink jet mechanism of a printer. In order to reduce the diameter of an opening for ejecting drug, the peripheral channel is required to decrease in size or change in physical shape. Therefore, the physical forces caused by forming droplets according to the principle of the ink jet system locally become significantly higher than the normal values in printing and higher than the shearing force or thermal energy caused by usual stirring. Consequently, the ejection of droplets tends to be difficult. Examples of the physical forces are pressure and shearing force. In an ink jet system, it is thought a load of 90 atmospheres is applied.

When an ink jet system is employed in the spray of drug, it is necessary that physical properties of a liquid to be ejected are adjusted to suitably control the state and amount of the minute droplets ejected from each ejection opening. That is, the liquid composition, such as the type and composition of a solvent and the solute content, constituting a liquid sample to be ejected is designed to obtain minute droplets at a desired volume.

Regarding pulmonary inhalation of droplets generated by a thermal ink jet system, a liquid composition containing a surface tension-controlling compound or a humectant is disclosed (refer to International Publication No. WO 02/094342). In the technology disclosed in this document, a surfactant or a water-soluble polymer such as polyethylene glycol is added to a solution for increasing surface tension, viscosity, and stability of protein in the solution formed into droplets by moisturizing function. However, detailed disclosure about ejection stability is not disclosed.

Furthermore, various technologies relating to an ejection mechanism based on the principle of the thermal ink jet system have been developed. In a usual ink jet printer head, the solution volume of the respective ejected droplets is about several picoliters, but a method and mechanism for ejecting extremely minute droplets in order of sub-picoliter or femtoliter has been developed (refer to Japanese Patent Application Laid-Open No. 2003-154655). For example, when a drug is applied to body cells of several micrometers in size, the above-mentioned extremely minute droplets are supposed to be required as the respective droplets to be ejected in some cases.

### SUMMARY OF THE INVENTION

It is another object of the present invention to provide a method for ejecting this ejection liquid and to provide a cartridge and an ejection device for treating respiratory disease by using this ejection liquid.

The ejection liquid according to the present invention is one to be ejected from an ejection opening of a device ejecting a drug used in treatment of respiratory disease. The ejection liquid includes: an active ingredient for treating respiratory disease; a surface-active compound represented by the following Formula (1):

R−A (1)

wherein R denotes a substituted or unsubstituted, saturated or unsaturated aliphatic chain of 3 or more and 30 or less carbon atoms or a saturated or unsaturated aliphatic derivative of 3 or more and 30 or less carbon atoms, and a includes at least one selected from the group consisting of a substituted or unsubstituted carboxylic acid group, a substituted or unsubstituted sulfonic acid group, a substituted or unsubstituted anion and its counter ion, a substituted or unsubstituted quaternary ammonium group, a substituted or unsubstituted cation and its counter ion, and a betaine skeleton), or an amine oxide or water-soluble nonionic compound having a substituted or unsubstituted, saturated or unsaturated aliphatic chain of 3 or more and 30 or less carbon atoms; and a liquid medium.

In a method for ejecting a liquid according to the present invention, the above-mentioned ejection liquid is ejected by an ink jet system from a device for ejecting a drug for treating respiratory disease.

A liquid ejection cartridge according to the present invention includes a tank for storing the above-mentioned ejection liquid and an ejection head.

An ejection device for treating respiratory disease according to the present invention includes a cartridge, and a channel and an opening for leading a liquid to be ejected from an ejection portion of a head of the cartridge to an inhalation region of a user.

According to the present invention, an ejection liquid which can be stably ejected from an extremely minute opening having a diameter of about several micrometers can be obtained by adding a particular surface-active compound to a liquid containing at least one active ingredient for treating respiratory disease.

Further features of the present invention will become apparent from the following description of exemplary embodiments with reference to the attached drawings.

### BRIEF DESCRIPTION OF THE DRAWINGS

FIG. 1 is an explanatory schematic view of an inhaler head cartridge unit.

FIG. 2 is a perspective view of an inhaler.

FIG. 3 is a perspective view of the inhaler of FIG. 2 in a state that an access cover is opened.

### DESCRIPTION OF THE EMBODIMENTS

An ejection liquid according to the present invention is a liquid composition containing at least one active ingredient for treating respiratory disease, a surfactant, and a liquid medium. This ejection liquid has characteristics which can be suitably used in a drug spray or drug inhaler for respiratory organs such as nose, throat, bronchus, bronchial tube, and lung. In particular, the ejection liquid has physical properties which are suitable for the ejection by an ink jet system. By using the ejection liquid of the present invention, for example, the ejection from an extremely minute opening having a diameter of about several micrometers of an ejection device in a thermal ink jet system can be stably achieved.

A liquid ejection cartridge includes at least a tank for storing this ejection liquid and a ejection head using an ink jet system. Further, a liquid ejection device used for inhaling a therapeutic drug can be structured by forming a channel and an opening at a liquid ejection portion of the ejection head of this liquid ejection cartridge. The channel lead a liquid ejected from the liquid ejection portion to an inhalation region of a user (patient).

The present invention will now be further described in detail.

In the present invention, the term “active ingredient (compound) for treating respiratory disease” refers to a compound used in treatment of various respiratory diseases such as asthma and chronic obstructive pulmonary disease (COPD), and typical examples of which include antitussives, respiratory stimulants, broncodilator, gargles, and expectorants. Specific examples of the active ingredient include cromoglycate, salbutamol, ipratropium, fenoterol, isoproterenol, trimetoquinol, procaterol, salmeterol, oxitropium, becromethasone propionate, bromhexine, acetylcysteine, budesonide, and fluticasone propionate. In addition, partially substituted derivatives of these compounds can be similarly used.

The content of the compound for treating respiratory disease in the ejection liquid is determined according to the purpose and use and is preferably 1 ng/ml or more and 200 mg/ml or less. The compound for treating respiratory disease may be used alone or in a combination of two or more.

The present inventors have conducted intensive studies and, as a result, have found the fact that when an ejection liquid is ejected as droplets from an ejection opening having a diameter of several micrometers based on the principle of an ink jet system, the droplets can be stably ejected by adding a particular surface-active compound to the liquid. The particular surface-active compound is a compound represented by the following Formula

(1) or an amine oxide or water-soluble nonionic compound having a substituted or unsubstituted, saturated or unsaturated aliphatic chain of 3 or more and 30 or less carbon atoms.

R−A (1)

In Formula (1), R denotes a substituted or unsubstituted, saturated or unsaturated aliphatic chain of 3 or more and 30 or less carbon atoms or a saturated or unsaturated aliphatic derivative of 3 or more and 30 or less carbon atoms, and a includes at least one selected from the group consisting of a substituted or unsubstituted carboxylic acid group, a substituted or unsubstituted sulfonic acid group, a substituted or unsubstituted anion and its counter ion, a substituted or unsubstituted quaternary ammonium group, a substituted or unsubstituted cation and its counter ion, and a betaine skeleton.

Any anions can be used as A in Formula (1), and A may include at least one anion selected from inorganic and/or organic anions. Specific examples of the anion include sulfonic acid, carboxylic acid, and phosphoric acid. The counter ion of the anion is a cation. Any cations can be used. The cation includes at least one cation selected from the group consisting of a monovalent metal ion, a metal oxide ion, and an organic cation. The counter ions may be the same or different. A preferable example of the monovalent metal ion is an alkali metal ion. Preferable examples of the metal oxide ion include VO₂⁺, RuO₄⁻, SnO₃²⁻, and TcO₄⁻. Preferably, the organic cation is selected from N⁺, P⁺ and S⁺. More preferably, the cation is a monovalent metal ion or quaternary ammonium ion.

Any cations can be used as A in Formula (1), and A may include at least one cation selected from inorganic and/or organic cations. The cation is specifically onium salt, more specifically ammonium salt. The counter ion of the cation is an anion. Any anions can be used. Preferable examples of the anion include halogen ion, chloride ion, bromide ion, iodide ion, fluoride ion, hydroxide ion, carboxylate ion, nitrate ion, phosphate ion, and sulfate ion. The counter ions may be the same or different.

The surface-active compound represented by Formula (1) preferably includes a substituted or unsubstituted alkyl group of 3 or more and 30 or less carbon atoms, and examples of which include alkyl amino acid, alkyl betaine, fatty acyl betaine, fatty acyl alkyl betaine, alkyl imidazolinium betaine, fatty acyl amino acid, alkyl sulfonic acid, alkylether amino acid salt, acylamino acid pyrroridone carboxylate, alkylamine oxide, fatty acyl amine oxide, fatty amino acid salt, ethylene diamine derivative, pyrroridone carboxylic acid, benzalkonium salt, benzethonium salt, fatty acyl polyoxyethylene sorbitan alkylate, and alkylbenzene sulfonic acid.

More preferable examples are the compounds having structures shown by the following Formulae (2) to (12).

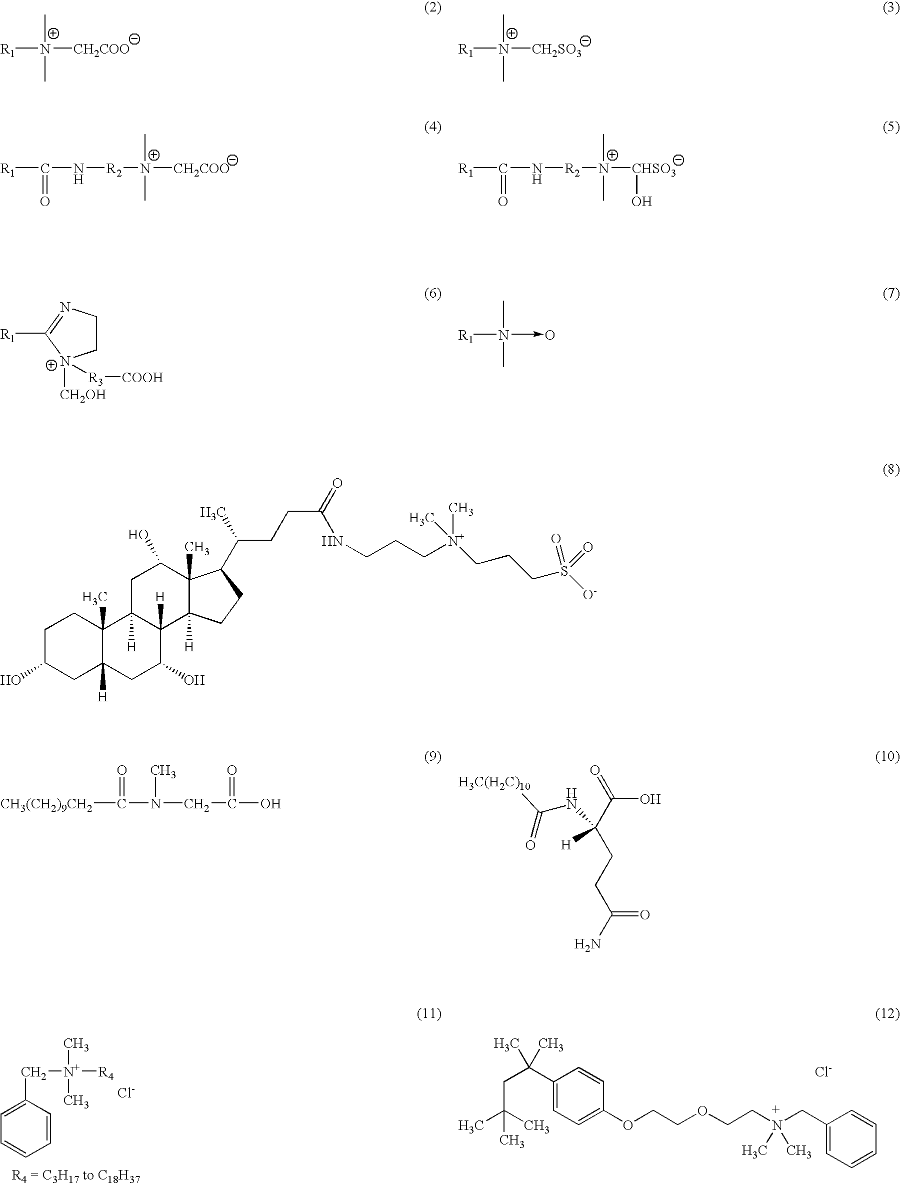

R in Formula (1) and R₁ in Formulae (2) to (7) each denote a substituted or unsubstituted, saturated or unsaturated aliphatic chain of 3 or more and 30 or less carbon atoms or a saturated or unsaturated aliphatic derivative of 3 or more and 30 or less carbon atoms. When the number of carbon atom is less than 3, the compound cannot have a surface-activating property. Conversely, when the number of carbon atom is higher than 30, the compound becomes a wax-lake state and, similarly, cannot have a surface-activating property.

R₂ and R₃ are each independently a substituted or unsubstituted, saturated or unsaturated alkylene chain of 1 or more and 6 or less carbon atoms.

Any derivatives of an aliphatic compound can be used as long as the physical properties and characteristics of the derivative are not largely altered from those of the aliphatic compound in a state not being a derivative. The ejection characteristic of a prescribed drug solution when a compound derivative is used may be higher than 50% of that of the intact compound.

Examples of the surface-active amine oxide include cocamidepropyl dimethylamine oxide and lauryl dimethylamine oxide.

Examples of the surface-active water-soluble nonionic compound include a polyoxyethylene sorbitan alkylate containing fatty acid, i.e., a polyoxyethylene sorbitan fatty acid ester. Particularly preferable examples are polyoxyethylene (20) sorbitan monolaurate, polyoxyethylene (4) sorbitan monoaurate, polyoxyethylene (20) sorbitan monopalmitate, polyoxyethylene (20) sorbitan monostearate, polyoxyethylene (20) sorbitan tristearate, polyoxyethylene (5) sorbitan monoaurate, and polyoxyethylene (20) sorbitan triaurate. Most preferable examples are polyoxyethylene (20) sorbitan monolaurate and polyoxyethylene (20) sorbitan monoaurate. Further, polyoxyethylene (20) sorbitan monolaurate and polyoxyethylene (20) sorbitan monoaurate are particularly preferable in pulmonary inhalation.

The grounds that the above-mentioned surface-active compounds highly stabilize the ejection are predicted as follows:

The compounds each contain a long-chain alkyl group and a hydrophilic functional group. Consequently, when a liquid is ejected as droplets from an opening having a diameter of several micrometers, the surface energy of the liquid is adequately controlled to obtain stable ejection property.

In the present invention, any surface-active compounds selected from the above can be used. In addition, some of the above-mentioned compounds have an antibacterial effect in addition to the surface-activating property. This is very useful added value also from the viewpoint of preservation stability of a drug. Preferable examples of the compound having both functions include benzalkonium chloride and benzethonium chloride.

The content of a surface-active compound added to an ejection liquid according to the present invention varies depending on the type of a drug coexistence with the compound in the ejection liquid. For example, when the drug is ipratropium bromide, the content of the surface-active compound is preferably 0.01 mass % or more and 20 mass % or less. When the content is lower than this range, the surface-activating property is insufficient. When the content is higher than the range, it may decrease the effect of a drug or cause adverse effects on a living body.

In the application of the present invention, one or more selected from antibacterial agents, germicides, and preservatives may be added to an ejection liquid according to need in order to eliminate or decrease influences of microorganisms. Examples of such ingredients include quaternary ammonium salts such as benzalkonium chloride and benzethonium chloride; phenol derivatives such as phenol, cresol, and anisole; benzoic acids such as benzoic acid and paraoxybenzoic acid; and sorbic acid.

In the application of the present invention, one or more selected from oil, glycerin, ethanol, urea, cellulose, polyethylene glycol, and alginate may be added to an ejection liquid according to need in order to increase the physical stability of the liquid during storage. In addition, in order to increase chemical stability, one or more antioxidants such as ascorbic acid, citric acid, cyclodextrin, and tocopherol may be added to the ejection liquid according to need.

Furthermore, for the purpose of adjusting the pH of an ejection liquid, a buffer may be added to the liquid. Examples of the buffer include ascorbic acid, citric acid, dilute hydrochloric acid, dilute sodium hydroxide, sodium hydrogen phosphate, sodium dihydrogen phosphate, potassium hydrogen phosphate, potassium dihydrogen phosphate, PBS, HEPES, and Tris buffet.

In addition, according to need, one or more isotonizing agents selected from aminoethyl sulfonic acid, potassium chloride, sodium chloride, glycerin, and sodium hydrogen carbonate may be added to the ejection liquid.

As a scenting agent and a flavoring agent, a sugar such as glucose or sorbitol, a sweetening agent such as aspartame, menthol, or a spice may be added. Further, not only hydrophilic chemicals but also hydrophobic chemicals or oils may be used.

In the present invention, various additives suitable to the intended use of an ejection liquid, for example, one or more selected from surface conditioner (surface tension controlling agent), viscosity controlling agents, solvents, and moisturizing agents may be added to the liquid in an appropriate volume according to need.

Specifically, a hydrophilic binder, a hydrophobic binder, a hydrophilic thickener, a hydrophobic thickener, a glycol derivative, an alcohol, a scenting agent, a flavoring agent, and an electrolyte are examples of compatible additives. These additives may be used alone or in a mixture thereof.

Preferably, the above-mentioned various additives are ones described in Pharmacopeia of various countries as minor constituents for medicinal purposes or administered ones for using in foods or cosmetics.

The blend ratios of the above-mentioned various additives vary depending on the type of a drug to be administered. Generally, the respective mass percentages of the additives are preferably 0.01% or more and 40% or less, more preferably 0.1% or more and 20% or less. In addition, the amount of an additive varies depending on the type and combination, but preferably is 0.5 part by mass or more and 200 parts by mass or less with respect to 1 part by mass of the drug from the viewpoint of the ejection characteristic.

The liquid medium for the ejection liquid according to the present invention may be mainly constituted of water. That is, the liquid medium may be constituted of water or a mixture solvent of water and a water-soluble organic solvent. That is, an ejection liquid can be prepared by adding the above-mentioned respective components to water so as to provide predetermined concentrations. The content of water is preferably 25% or more.

A liquid ejection device according to the present invention has a structure suitable as a spray device for drug inhalation into respiratory system. The liquid ejection device employs a system for ejecting a liquid from an ejection opening. The liquid ejection system is preferably an ink jet system. As the ink jet system, various systems are known according to the type of ejection energy. The typical examples are a system using a piezoelectric element and a system using a heater element (thermal ink jet system). As mentioned above, among these systems, a thermal ink jet system is preferable. For example, preferably, the liquid ejection device includes a ejection head which can ejection minute droplets of a liquid agent containing a medicinal ingredient by a thermal ink jet system and has a structure which can independently drive a large number of liquid agent-ejecting units constituting this head. In such a case, electric lines and wiring are united. The electric lines connect a plurality of control signals which are necessary for independently driving the respective liquid agent-ejecting units. The wiring connects among the liquid agent-ejecting units. In addition, an integrated liquid ejection cartridge including a tank for storing the liquid agent and a liquid channel as a means for supplying a liquid agent from the tank to the ejection head based on the principle of a thermal ink jet system is preferable. In the present invention, among an ink jet system as usually used in a printing fields an embodiment capable of providing a thermal energy by using an electrothermal transducer is described as “thermal ink jet system” and an embodiment capable of providing a mechanical energy by using an electrothermal transducer is described as “piezo ink jet system”. These terms are employed for a liquid for treating respiratory disease, but such terms are merely expressed providing a liquid with ejection energy according to the principle of the ink jet system.

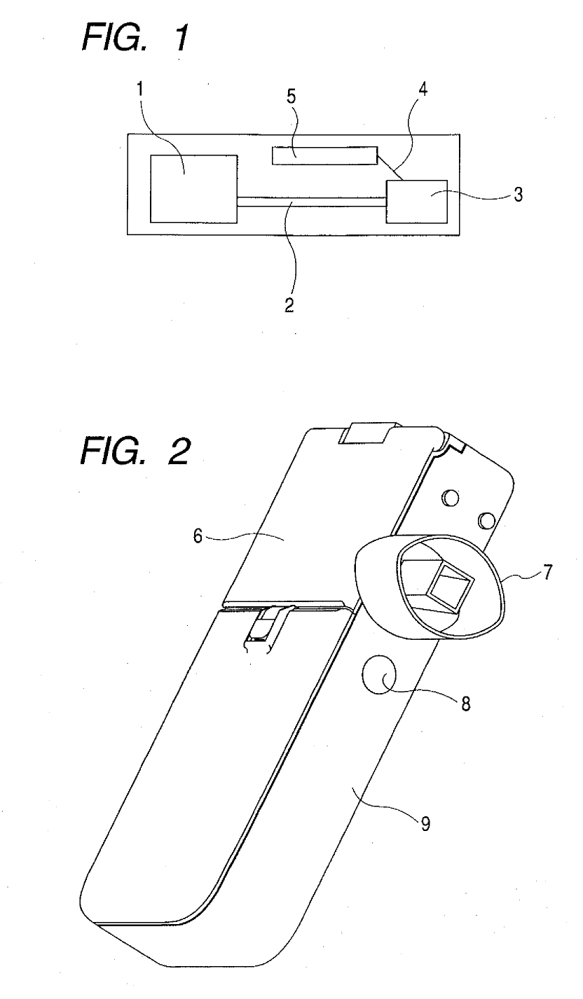

FIG. 1 schematically illustrates the entire structure of a liquid ejection cartridge according to an embodiment of the present invention. The cartridge shown in FIG. 1 is fabricated on one substrate by integrally disposing a head 3 for spraying a liquid agent, a tank 1 for storing the liquid agent, and a liquid channel 2 for leading the liquid agent from the tank 1 to the head 3. The head 3 exchanges driving signals and controlling signals with a controller for controlling the driving of each liquid agent-ejecting unit of the head 3 through electric lines 5 which are connected to internal wiring 4.

As an example of the head 3, an ejection head disclosed in Japanese Patent Application Laid-Open No. 2003-154655 which can eject extremely minute droplets each in order of sub-picoliter or femtoliter and also superior in the controlling property is preferable.

In the embodiment shown in FIG. 1, one kind of a liquid agent is sprayed and therefore one tank for the liquid agent is provided. When two or more kinds of liquid agents are sprayed, plural tanks for the respective liquid agents are optionally provided and thermal ink jet heads also deal with it by integrating a plurality of kinds of liquid agent-ejecting units.

The liquid ejection device according to the present invention can be particularly suitably applied to a system in which a process for exchanging a liquid agent into minute droplets and a process for mixing the sprayed minute droplets with a carrier gas flow are separately carried out. Such a system is a typical method for spraying. By the ejection device employing such a system, the ejection device can effectively utilize the advantage obtained by separating the ejection process into a process for exchanging into droplets and a process for mixing the droplets with a gas flow. That is, when a liquid agent containing a drug compound at a predetermined concentration corresponding to the treatment purpose is sprayed as an ejection liquid and is inhaled by a subject to be administered with the liquid agent, the volume (dosage per unit volume) of the drug compound contained in the gas to be inhaled can be optionally adjusted. Further, the device can be reduced in size so as to be portable by a user by utilizing an ejection head based on the principle of ink jet in which openings for minute droplets are arranged at a high density per unit area, as a mechanism for spraying a liquid agent. According to this liquid ejection device, the drug compound concentration in the gas flow can be changed only by changing the ejection rate.

In pulmonary inhalation, preferably, the liquid ejection device can eject droplets having an average particle size of 0.5 μm or more and 8 μm or less, more preferably 1 μm or more and 5 μm or less, and further preferably 2 μm or more and 5 μm or less and having a narrow particle size distribution.

The liquid ejection device according to the present invention can be suitably used as an inhalation device (inhaler) of a medicinal ingredient (drug) used in treatment of respiratory disease by employing a cartridge type device functioning as a controller of the ejection rate. This inhaler is designed to be portable for a user and can eject a predetermined amount of a drug as droplets having a uniform particle size so that a user can inhale a constant volume of the drug.

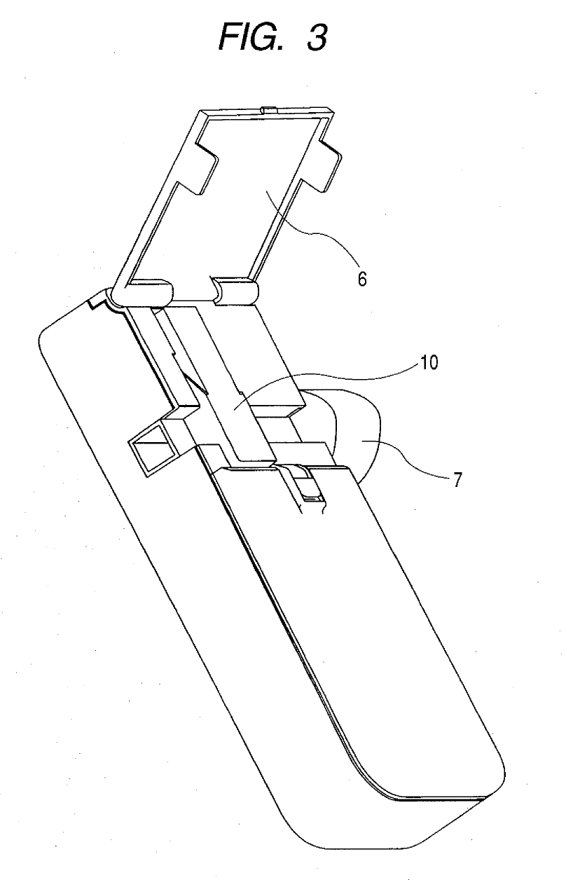

An inhaler according to the present invention will be roughly described by referring to FIGS. 2 and 3.

FIG. 2 is a cross-sectional view of an appearance of the inhaler. A housing includes an inhaler body 9 and an access cover 6. FIG. 3 is a view when the access cover 6 is opened and thereby a head cartridge unit 10 can be seen. By the operation by a user for inhalation, the air enters into a mouthpiece 7 from an air intake and is mixed with a drug ejected from an ejection opening disposed to a head (not shown) of the head cartridge unit 10. The resulting mixture fluid is moved toward the outlet of the mouthpiece 7 which is formed into a shape to be held by the mouth of a user. The user can effectively inhale the droplets of a drug solution ejected from the liquid-ejecting portion of the head cartridge unit by inserting the end of the mouthpiece 7 into the mouth and holding it by the teeth and drawing in a breath. In addition, inside the housing, a controller (not shown) of the head cartridge unit and a power supply (battery) (not shown) are disposed. As shown in FIG. 1, the head cartridge unit is integrally provided with a liquid-ejecting portion and a liquid agent-storing tank and is exchangeable by opening the access cover 6.

As shown in FIG. 3, the head cartridge unit 10 is disposed at a midpoint of a tubular air channel for leading the air inflowing from the air inlet into the mouthpiece 7. The liquid agent to be formed into minute droplets on the basis of the principle of a thermal ink jet system and sprayed at the head of the head cartridge unit 10 is mixed with the air flowing in this, tubular air channel. This inhaler employs a system in which the air inflows from the air inlet by that a user holds the mouthpiece 7 by the mouth and inhales a gas. That is, the structure of the air inlet portion corresponds to an inhaling mechanism which allows a subject to be administered with a liquid agent as a gas generated by a spray mechanism and in which minute droplet mist of the liquid agent is suspended.

By employing the structure shown in FIG. 3, the minute droplets of a liquid agent are sprayed by inhalation and spontaneously reach throat and inside bronchus of a subject to be administered. Therefore, the amount (dose) of the liquid agent to be sprayed is controlled independently of the volume of inhaled air. Specifically, the head of the head cartridge unit 10 has a structure using a minute droplet-ejection head disclosed in Japanese Patent Application Laid-Open No. 2003-154655 and ejecting droplets having an average particle size of about 3 μm. The diameter of opening provided to the head is 0.5 μm or more and 8 μm or less, preferably 1 μm or more and 5 μm or less, and further preferably 2 μm or more and 5 μm or less.

### EXAMPLES

The present invention will now be described in further detail with reference to Examples. These Examples are shown only for better understanding of the present invention and do not limit the scope of the invention. In Examples, “%” denotes % by mass.

### Examples 1 to 234 and Comparative Examples 1 to 12

(Formation of Droplets of Drug Solution According to Principle of Thermal Ink Jet System)

Ejection liquids were each prepared by preliminarily dissolving a drug compound for treating respiratory disease in purified water at an appropriate concentration, adding a surface-active compound thereto under stirring, and then adjusting the content of each ingredient to a desired level with purified water.

The resulting liquid was measured for the particle size and particle size distribution using a laser diffraction particle size analyzer (Spraytec: Malvern) to confirm a droplet size distribution with a sharp peak. The droplet size was also measured.

The head cartridge was filled with the prepared ejection liquid and was connected to an ejection controller. Then, the ejection was carried out at a frequency of 20 kHz and a voltage of 12 V for 1 sec. After an interval of 3 sec, the next ejection was carried out. This process was repeated for 200 times, and it was visually confirmed whether the ejection was performed or not. The ejection was evaluated as excellent when the ejection was performed not less than 100 times, good when the ejection was performed 15 times or more but less than 100 times, and poor when the ejection was performed less than 15 times. The ejection liquid was subjected to HPLC analysis before and after the ejecting to confirm a change in the composition of the ejection liquid (Measurement conditions were device: JASCO corporation, column: YMC-Pack Diol-200, 500×8.0 mm ID, eluent: 0.1 M KH₂PO₄—K₂HPO₄ (pH 7.0) containing 0.2 M NaCl, flow rate: 0.7 ml/min, temperature: 25° C., and detection: UV at 215 nm).

As Comparative Examples, ejection liquids containing purified water, ethanol, various drug solutions, and substances other than compounds according to the present invention were prepared and subjected to an ejection test as in the Examples. Table 1 shows the prescription investigated in Examples and Comparative Examples and results thereof (droplet size and ejection characteristic).

TABLE 1

| | | | | | Nozzle | | Ejection |
|---|---|---|---|---|---|---|---|
| | Drug Compound | | Surface Active Compound | | Diam- | Particle | Character- |
| | Type | Concentration | Type | Concentration | eter | Size | istic |
| Example 1 | Ipratropium bromide | 0.03% | Benzalkonium chloride | 1% | 3 μm | 3 μm | Excellent |
| Example 2 | Ipratropium bromide | 0.03% | Lauroylsarcosine | 1% | 3 μm | 3 μm | Excellent |
| Example 3 | Ipratropium bromide | 0.03% | Benzethonium chloride | 1% | 3 μm | 3 μm | Excellent |
| Example 4 | Ipratropium bromide | 0.03% | Lauramide propyl betaine | 1% | 3 μm | 3 μm | Excellent |
| Example 5 | Ipratropium bromide | 0.03% | Cocoyl glutamatc | 1% | 3 μm | 3 μm | Excellent |
| Example 6 | Ipratropium bromide | 0.03% | CHAPS | 1% | 3 μm | 3 μm | Excellent |
| Example 7 | Ipratropium bromide | 0.03% | Dodecylbenzenesulfonic acid | 1% | 3 μm | 3 μm | Excellent |
| Example 8 | Ipratropium bromide | 0.03% | SDS | 1% | 3 μm | 3 μm | Excellent |
| Example 9 | Ipratropium bromide | 0.03% | Cocamidopropyl betaine | 1% | 3 μm | 3 μm | Excellent |
| Example 10 | Ipratropium bromide | 0.03% | Alkylcarboxymethylhydroxyethyl | 1% | 3 μm | 3 μm | Excellent |
| | | | imidazolinium betaine | | | | |
| Example 11 | Ipratropium bromide | 0.03% | Cocoyl glycine | 1% | 3 μm | 3 μm | Excellent |
| Example 12 | Ipratropium bromide | 0.03% | Cocyl carboxymethylhydroxyethyl | 1% | 3 μm | 3 μm | Excellent |
| | | | ethylenediamine | | | | |
| Example 13 | Ipratropium bromide | 0.03% | Laurylaminopropionic acid | 1% | 3 μm | 3 μm | Excellent |
| Example 14 | Ipratropium bromide | 0.03% | Lauryldimethylaminoacetic acid betaine | 1% | 3 μm | 3 μm | Excellent |
| Example 15 | Ipratropium bromide | 0.03% | Lauryl betaine | 1% | 3 μm | 3 μm | Excellent |
| Example 16 | Ipratropium bromide | 0.03% | Cocoyl alanine | 1% | 3 μm | 3 μm | Excellent |
| Example 17 | Ipratropium bromide | 0.03% | Coconut oil fatty acid arginine salt | 1% | 3 μm | 3 μm | Excellent |
| Example 18 | Ipratropium bromide | 0.03% | Cocamidopropylhydroxysulfobetaine | 1% | 3 μm | 3 μm | Excellent |
| Example 19 | Ipratropium bromide | 0.03% | Cocamidopropyldimethylamine oxide | 1% | 3 μm | 3 μm | Excellent |
| Example 20 | Ipratropium bromide | 0.03% | Laurylhydroxysulfobetaine | 1% | 3 μm | 3 μm | Excellent |
| Example 21 | Ipratropium bromide | 0.03% | Lauryldimethylamine oxide | 1% | 3 μm | 3 μm | Excellent |
| Example 22 | Ipratropium bromide | 0.03% | Cocoylarginine ethylpyrroridone | 1% | 3 μm | 3 μm | Excellent |
| | | | carboxylate | | | | |
| Example 23 | Ipratropium bromide | 0.03% | Alkyloxyhydroxypropylarginine | 1% | 3 μm | 3 μm | Excellent |
| | | | hydrochloride | | | | |
| Example 24 | Ipratropium bromide | 0.03% | Pyrroridone carboxylate | 5% | 3 μm | 3 μm | Excellent |
| Example 25 | Ipratropium bromide | 0.03% | tween20 | 5% | 3 μm | 3 μm | Excellent |
| Example 26 | Ipratropium bromide | 0.03% | tween80 | 5% | 3 μm | 3 μm | Excellent |
| Example 27 | Fenoterol hydrobromide | 0.15% | Benzalkonium chloride | 1% | 3 μm | 3 μm | Excellent |
| Example 28 | Fenoterol hydrobromide | 0.15% | Lauroylsarcosine | 1% | 3 μm | 3 μm | Excellent |
| Example 29 | Fenoterol hydrobromide | 0.15% | Benzethonium chloride | 1% | 3 μm | 3 μm | Excellent |
| Example 30 | Fenoterol hydrobromide | 0.15% | Lauramide propyl betaine | 1% | 3 μm | 3 μm | Excellent |
| Example 31 | Fenoterol hydrobromide | 0.15% | Cocoyl glutamate | 1% | 3 μm | 3 μm | excellent |
| Example 32 | Fenoterol hydrobromide | 0.15% | CHAPS | 1% | 3 μm | 3 μm | Excellent |
| Example 33 | Fenoterol hydrobromide | 0.15% | Dodecylbenzenesulfonic acid | 1% | 3 μm | 3 μm | Excellent |
| Example 34 | Fenoterol hydrobromide | 0.15% | SDS | 1% | 3 μm | 3 μm | Excellent |
| Example 35 | Fenoterol hydrobromide | 0.15% | Cocamidopropyl betaine | 1% | 3 μm | 3 μm | Excellent |
| Example 36 | Fenoterol hydrobromide | 0.15% | Alkylcarboxymethylhydroxyethyl | 1% | 3 μm | 3 μm | Excellent |
| | | | imidazolinium betaine | | | | |
| Exampel 37 | Fenoterol hydrobromide | 0.15% | Cocoyl glycine | 1% | 3 μm | 3 μm | Excellent |
| Example 38 | Fenoterol hydrobromide | 0.15% | Cocoyl carboxymethylhydroxyethyl | 1% | 3 μm | 3 μm | Excellent |
| | | | ethylenediamine | | | | |
| Example 39 | Fenoterol hydrobromide | 0.15% | Laurylaminopropionic acid | 1% | 3 μm | 3 μm | Excellent |
| Example 40 | Fenoterol hydrobromide | 0.15% | Lauryldimethylaminoacetic acid betaine | 1% | 3 μm | 3 μm | Excellent |
| Example 41 | Fenoterol hydrobromide | 0.15% | Lauryl betaine | 1% | 3 μm | 3 μm | Excellent |
| Example 42 | Fenoterol hydrobromide | 0.15% | Cocoyl alanine | 1% | 3 μm | 3 μm | Excellent |
| Example 43 | Fenoterol hydrobromide | 0.15% | Coconut oil fatty acid arginine salt | 1% | 3 μm | 3 μm | Excellent |
| Example 44 | Fenoterol hydrobromide | 0.15% | Lauramidopropylhydroxysulfobetaine | 1% | 3 μm | 3 μm | Exellent |
| Example 45 | Fenoterol hydrobromide | 0.15% | Lauramidopropyldimethylamine oxide | 1% | 3 μm | 3 μm | Excellent |
| Example 46 | Fenoterol hydrobromide | 0.15% | Laurylhydroxysulfobetaine | 1% | 3 μm | 3 μm | Excellent |
| Example 47 | Fenoterol hydrobromide | 0.15% | Lauryldimethylamine oxide | 1% | 3 μm | 3 μm | Excellent |
| Example 48 | Fenoterol hydrobromide | 0.15% | Cocoylarginine ethylpyrroridone | 1% | 3 μm | 3 μm | Excellent |
| | | | carboxylate | | | | |
| Example 49 | Fenoterol hydrobromide | 0.15% | Alkyloxyhydroxypropylarginine | 1% | 3 μm | 3 μm | Excellent |
| | | | hydrochloride | | | | |
| Example 50 | Fenoterol hydrobromide | 0.15% | Pyrroridone carboxylate | 5% | 3 μm | 3 μm | Excellent |
| Example 51 | Fenoterol hydrobromide | 0.15% | tween20 | 5% | 3 μm | 3 μm | Excellent |
| Example 52 | Fenoterol hydrobromide | 0.15% | tween80 | 5% | 3 μm | 3 μm | Excellent |
| Example 53 | Sodium salbutamol sulfate | 1.00% | Benzalkonium chloride | 1% | 3 μm | 3 μm | Excellent |
| Example 54 | Sodium salbutamol sulfate | 1.00% | Lauroylsarcosine | 1% | 3 μm | 3 μm | Excellent |
| Example 55 | Sodium salbutamol sulfate | 1.00% | Benzethonium chloride | 1% | 3 μm | 3 μm | Excellent |
| Example 56 | Sodium salbutamol sulfate | 1.00% | Lauramide propyl betaine | 1% | 3 μm | 3 μm | Excellent |
| Example 57 | Sodium salbutamol sulfate | 1.00% | Cocoyl glutamate | 1% | 3 μm | 3 μm | Excellent |
| Example 58 | Sodium salbutamol sulfate | 1.00% | CHAPS | 1% | 3 μm | 3 μm | Excellent |
| Example 59 | Sodium salbutamol sulfate | 1.00% | Dodecylbenzenesulfonic acid | 1% | 3 μm | 3 μm | Excellent |
| Example 60 | Sodium salbutamol sulfate | 1.00% | SDS | 1% | 3 μm | 3 μm | Excellent |
| Example 61 | Sodium salbutamol sulfate | 1.00% | Cocamidopropyl betaine | 1% | 3 μm | 3 μm | Excellent |
| Example 62 | Sodium salbutamol sulfate | 1.00% | Alkylcarboxymethylhydroxyethyl | 1% | 3 μm | 3 μm | Excellent |
| | | | imidazolinium betaine | | | | |
| Example 63 | Sodium salbutamol sulfate | 1.00% | Cocoyl glycine | 1% | 3 μm | 3 μm | Excellent |
| Example 64 | Sodium salbutamol sulfate | 1.00% | Cocoyl carboxymethylhydroxyethyl | 1% | 3 μm | 3 μm | Excellent |
| | | | ethylenediamine | | | | |
| Example 65 | Sodium salbutamol sulfate | 1.00% | Laurylaminopropionic acid | 1% | 3 μm | 3 μm | Excellent |
| Example 66 | Sodium salbutamol sulfate | 1.00% | Lauryldimethylaminoacetic acid betaine | 1% | 3 μm | 3 μm | Excellent |
| Example 67 | Sodium salbutamol sulfate | 1.00% | Lauryl betaine | 1% | 3 μm | 3 μm | Excellent |
| Example 68 | Sodium salbutamol sulfate | 1.00% | Cocoyl alanine | 1% | 3 μm | 3 μm | Excellent |
| Example 69 | Sodium salbutamol sulfate | 1.00% | Coconut oil fatty acid arginine salt | 1% | 3 μm | 3 μm | |
| Example 70 | Sodium salbutamol sulfate | 1.00% | Cocamidopropylhydroxysulfobetaine | 1% | 3 μm | 3 μm | Excellent |
| Example 71 | Sodium salbutamol sulfate | 1.00% | Cocamidopropyldimethylamine oxide | 1% | 3 μm | 3 μm | Excellent |
| Example 72 | Sodium salbutamol sulfate | 1.00% | Laurylhydroxysulfobetaine | 1% | 3 μm | 3 μm | Excellent |
| Example 73 | Sodium salbutamol sulfate | 1.00% | Lauryldimethylamine oxide | 1% | 3 μm | 3 μm | Excellent |
| Example 74 | Sodium salbutamol sulfate | 1.00% | Cocoylarginine ethylpyrroridone | 1% | 3 μm | 3 μm | Excellent |
| | | | carboxylate | | | | |
| Example 75 | Sodium salbutamol sulfate | 1.00% | Alkyloxyhydroxypropylarginine | 1% | 3 μm | 3 μm | Excellent |
| | | | hydrochloride | | | | |
| Example 76 | Sodium salbutamol sulfate | 1.00% | Pyrroridone carboxylate | 5% | 3 μm | 3 μm | Excellent |
| Example 77 | Sodium salbutamol sulfate | 1.00% | tween20 | 5% | 3 μm | 3 μm | Excellent |
| Example 78 | Sodium salbutamol sulfate | 1.00% | tween80 | 5% | 3 μm | 3 μm | Excellent |
| Example 79 | Sodium cromoglycate | 1.00% | Benzalkonium chloride | 1% | 3 μm | 3 μm | Excellent |
| Example 80 | Sodium cromoglycate | 1.00% | Lauroylsarcosine | 1% | 3 μm | 3 μm | Excellent |
| Example 81 | Sodium cromoglycate | 1.00% | Benzethonium chloride | 1% | 3 μm | 3 μm | Excellent |
| Example 82 | Sodium cromoglycate | 1.00% | Lauramide propyl betaine | 1% | 3 μm | 3 μm | Excellent |
| Example 83 | Sodium cromoglycate | 1.00% | Cocoyl glutamate | 1% | 3 μm | 3 μm | Excellent |
| Example 84 | Sodium cromoglycate | 1.00% | CHAPS | 1% | 3 μm | 3 μm | Excellent |
| Example 85 | Sodium cromoglycate | 1.00% | Dodecylbenzenesulfonic acid | 1% | 3 μm | 3 μm | Excellent |
| Example 86 | Sodium cromoglycate | 1.00% | SDS | 1% | 3 μm | 3 μm | Excellent |
| Example 87 | Sodium cromoglycate | 1.00% | Cocamidopropyl betaine | 1% | 3 μm | 3 μm | Excellent |
| Example 88 | Sodium cromoglycate | 1.00% | Alkylcarboxymethylhydroxyethyl | 1% | 3 μm | 3 μm | Excellent |
| | | | imidazolinium betaine | | | | |
| Example 89 | Sodium cromoglycate | 1.00% | Cocoyl glycine | 1% | 3 μm | 3 μm | Excellent |
| Example 90 | Sodium cromoglycate | 1.00% | Cocoyl carboxymethylhydroxyethyl | 1% | 3 μm | 3 μm | Excellent |
| | | | ethylenediamine | | | | |
| Example 91 | Sodium cromoglycate | 1.00% | Laurylaminopropionic acid | 1% | 3 μm | 3 μm | Excellent |
| Example 92 | Sodium cromoglycate | 1.00% | Lauryldimethylaminoacetic acid betaine | 1% | 3 μm | 3 μm | Excellent |
| Example 93 | Sodium cromoglycate | 1.00% | Lauryl betaine | 1% | 3 μm | 3 μm | Excellent |
| Example 94 | Sodium cromoglycate | 1.00% | Cocoyl alanine | 1% | 3 μm | 3 μm | Excellent |
| Example 95 | Sodium cromoglycate | 1.00% | Coconut oil fatty acid arginine salt | 1% | 3 μm | 3 μm | Excellent |
| Example 96 | Sodium cromoglycate | 1.00% | Cocamidopropylhydroxysulfobetaine | 1% | 3 μm | 3 μm | Excellent |
| Example 97 | Sodium cromoglycate | 1.00% | Cocamidopropyldimethylamine oxide | 1% | 3 μm | 3 μm | Excellent |
| Example 98 | Sodium cromoglycate | 1.00% | Laurylhydroxysulfobetaine | 1% | 3 μm | 3 μm | Excellent |
| Example 99 | Sodium cromoglycate | 1.00% | Lauryldimethylamine oxide | 1% | 3 μm | 3 μm | Excellent |
| Example 100 | Sodium cromoglycate | 1.00% | Cocoylarginine ethylpyrroridone | 1% | 3 μm | 3 μm | Excellent |
| | | | carboxylate | | | | |
| Example 101 | Sodium cromoglycate | 1.00% | Alkloxyhydroxypropylarginine | 1% | 3 μm | 3 μm | Excellent |
| | | | hydrochloride | | | | |
| Example 102 | Sodium cromoglycate | 1.00% | Pyrroridone carboxylate | 5% | 3 μm | 3 μm | Excellent |
| Example 103 | Sodium cromoglycate | 1.00% | tween20 | 5% | 3 μm | 3 μm | Excellent |
| Example 104 | Sodium cromoglycate | 1.00% | tween80 | 5% | 3 μm | 3 μm | Excellent |
| Example 105 | Acetylcysteine | 0.50% | Benzalkonium chloride | 1% | 6 μm | 6.1 μm | Excellent |
| Example 106 | Acetylcysteine | 0.50% | Lauroylsarcosine | 1% | 6 μm | 6.1 μm | Excellent |
| Example 107 | Acetylcysteine | 0.50% | Benzethonium chloride | 1% | 6 μm | 6.1 μm | Excellent |
| Example 108 | Acetylcysteine | 0.50% | Lauramide propyl betaine | 1% | 6 μm | 6.1 μm | Excellent |
| Example 109 | Acetylcysteine | 0.50% | Cocoyl glutamate | 1% | 6 μm | 6.1 μm | Excellent |
| Example 110 | Acetylcysteine | 0.50% | CHAPS | 1% | 6 μm | 6.1 μm | Excellent |
| Example 111 | Acetylcysteine | 0.50% | Dodecylbenzenesulfonic acid | 1% | 6 μm | 6.1 μm | Excellent |
| Example 112 | Acetylcysteine | 0.50% | SDS | 1% | 6 μm | 6.1 μm | Excellent |
| Example 113 | Acetylcysteine | 0.50% | Cocamidopropyl betaine | 1% | 6 μm | 6.1 μm | Excellent |
| Example 114 | Acetylcysteine | 0.50% | Alkylcarboxymethylhydroxyethyl | 1% | 6 μm | 6.1 μm | Excellent |
| | | | imidazolinium betaine | | | | |
| Example 115 | Acetylcysteine | 0.50% | Cocoyl glycine | 1% | 6 μm | 6.1 μm | Excellent |
| Example 116 | Acetylcysteine | 0.50% | Cocoyl carboxymethylhydroxyethyl | 1% | 6 μm | 6.1 μm | Excellent |
| | | | ethylenediamine | | | | |
| Example 117 | Acetylcysteine | 0.50% | Laurylaminopropionic acid | 1% | 6 μm | 6.1 μm | Excellent |
| Example 118 | Acetylcysteine | 0.50% | Lauryldimethylaminoacetic acid betaine | 1% | 6 μm | 6.1 μm | Excellent |
| Example 119 | Acetylcysteine | 0.50% | Lauryl betaine | 1% | 6 μm | 6.1 μm | Excellent |
| Example 120 | Acetylcysteine | 0.50% | Cocoyl alanine | 1% | 6 μm | 6.1 μm | Excellent |
| Example 121 | Acetylcysteine | 0.50% | Coconut oil fatty arginine salt | 1% | 6 μm | 6.1 μm | Excellent |
| Example 122 | Acetylcysteine | 0.50% | Cocamidopropylhydroxysulfobetaine | 1% | 6 μm | 6.1 μm | Excellent |
| Example 123 | Acetylcysteine | 0.50% | Cocamidopropyldimethylamine oxide | 1% | 6 μm | 6.1 μm | Excellent |
| Example 124 | Acetylcysteine | 0.50% | Laurylhydroxysulfobetaine | 1% | 6 μm | 6.1 μm | Excellent |
| Example 125 | Acetylcysteine | 0.50% | Lauryldimethylamine oxide | 1% | 6 μm | 6.1 μm | Excellent |
| Example 126 | Acetylcysteine | 0.50% | Cocoylarginine ethylpyrroridone | 1% | 6 μm | 6.1 μm | Excellent |
| | | | carboxylate | | | | |
| Example 127 | Acetylcysteine | 0.50% | Alkyloxyhydroxypropylarginine | 1% | 6 μm | 6.1 μm | excellent |
| | | | hydrochloride | | | | |
| Example 128 | Acetylcysteine | 0.50% | Pyrroridone carboxylate | 5% | 6 μm | 6.1 μm | Excellent |
| Example 129 | Acetylcysteine | 0.50% | tween20 | 5% | 6 μm | 6.1 μm | Excellent |
| Example 130 | Acetylcysteine | 0.50% | tween80 | 5% | 6 μm | 6.1 μm | Excellent |
| Example 131 | Fluticasone propionate | 0.05% | Benzalkonium chloride | 1% | 6 μm | 6.1 μm | Excellent |
| Example 132 | Fluticasone propionate | 0.05% | Laurylsarcosine | 1% | 6 μm | 6.1 μm | Excellent |
| Example 133 | Fluticasone propionate | 0.05% | Benzethonium chloride | 1% | 6 μm | 6.1 μm | Excellent |
| Example 134 | Fluticasone propionate | 0.05% | Lauramide propyl betaine | 1% | 6 μm | 6.1 μm | Excellent |
| Example 135 | Fluticasone propionate | 0.05% | Cocoyl glutamate | 1% | 6 μm | 6.1 μm | Excellent |
| Example 136 | Fluticasone propionate | 0.05% | CHAPS | 1% | 6 μm | 6.1 μm | Excellent |
| Example 137 | Fluticasone propionate | 0.05% | Dodecylbenzenesulfonic acid | 1% | 6 μm | 6.1 μm | Excellent |
| Example 138 | Fluticasone propionate | 0.05% | SDS | 1% | 6 μm | 6.1 μm | Excellent |
| Example 139 | Fluticasone propionate | 0.05% | Cocamidopropyl betaine | 1% | 6 μm | 6.1 μm | Excellent |
| Example 140 | Fluticasone propionate | 0.05% | Alkylcarboxymethylhydroxyethyl | 1% | 6 μm | 6.1 μm | Excellent |
| | | | imidazolinium betaine | | | | |
| Example 141 | Fluticasone propionate | 0.05% | Cocoyl glycine | 1% | 6 μm | 6.1 μm | Excellent |
| Example 142 | Fluticasone propionate | 0.05% | Cocoyl carboxymethylhydroxyethyl | 1% | 6 μm | 6.1 μm | Excellent |
| | | | ethylenediamine | | | | |
| Example 143 | Fluticasone propionate | 0.05% | Laurylaminopropionic acid | 1% | 6 μm | 6.1 μm | Excellent |
| Example 144 | Fluticasone propionate | 0.05% | Lauryldimethylaminoacetic acid betaine | 1% | 6 μm | 6.1 μm | Excellent |
| Example 145 | Fluticasone propionate | 0.05% | Lauryl betaine | 1% | 6 μm | 6.1 μm | Excellent |
| Example 146 | Fluticasone propionate | 0.05% | Cocoyl alanine | 1% | 6 μm | 6.1 μm | Excellent |
| Example 147 | Fluticasone propionate | 0.05% | Coconut oil fatty arginine salt | 1% | 6 μm | 6.1 μm | Excellent |
| Example 148 | Fluticasone propionate | 0.05% | Cocamidopropylhydroxysulfobetaine | 1% | 6 μm | 6.1 μm | Excellent |
| Example 149 | Fluticasone propionate | 0.05% | Cocamidopropyldimethylamine oxide | 1% | 6 μm | 6.1 μm | Excellent |
| Example 150 | Fluticasone propionate | 0.05% | Laurylhydroxysulfobetaine | 1% | 6 μm | 6.1 μm | Excellent |
| Example 151 | Fluticasone propionate | 0.05% | Lauryldimethylamine oxide | 1% | 6 μm | 6.1 μm | Excellent |
| Example 152 | Fluticasone propionate | 0.05% | Cocoylarginine ethylpyrroridone | 1% | 6 μm | 6.1 μm | Excellent |
| | | | carboxylate | | | | |
| Example 153 | Fluticasone propionate | 0.05% | Alkyloxyhydroxypropylarginine | 1% | 6 μm | 6.1 μm | Excellent |
| | | | hydrochloride | | | | |
| Example 154 | Fluticasone propionate | 0.05% | Pyrroridone carboxylate | 5% | 6 μm | 6.1 μm | Excellent |
| Example 155 | Fluticasone propionate | 0.05% | tween20 | 5% | 6 μm | 6.1 μm | Excellent |
| Example 156 | Fluticasone propionate | 0.05% | tween80 | 5% | 6 μm | 6.1 μm | Excellent |
| Example 157 | Becromethasone propionate | 0.05% | Benzalkonium chloride | 1% | 3 μm | 3.1 μm | Excellent |
| Example 158 | Becromethasone propionate | 0.05% | Lauroylsarcosine | 1% | 3 μm | 3.1 μm | Excellent |
| Example 159 | Becromethasone propionate | 0.05% | Benzethonium chloride | 1% | 3 μm | 3.1 μm | Excellent |
| Example 160 | Becromethasone propionate | 0.05% | Lauramide propyl betaine | 1% | 3 μm | 3.1 μm | Excellent |
| Example 161 | Becromethasone propionate | 0.05% | Cocoyl glutamate | 1% | 3 μm | 3.1 μm | Excellent |
| Example 162 | Becromethasone propionate | 0.05% | CHAPS | 1% | 3 μm | 3.1 μm | Excellent |
| Example 163 | Becromethasone propionate | 0.05% | Dodecylbenzenesulfonic acid | 1% | 3 μm | 3.1 μm | Excellent |
| Example 164 | Becromethasone propionate | 0.05% | SDS | 1% | 3 μm | 3.1 μm | Excellent |
| Example 165 | Becromethasone propionate | 0.05% | Cocamidopropyl betaine | 1% | 3 μm | 3.1 μm | Excellent |
| Example 166 | Becromethasone propionate | 0.05% | Alkylcarboxymethylhydroxyethyl | 1% | 3 μm | 3.1 μm | Excellent |
| | | | imidazolinium betaine | | | | |
| Example 167 | Becromethasone propionate | 0.05% | Cocoyl glycine | 1% | 3 μm | 3.1 μm | Excellent |
| Example 168 | Becromethasone propionate | 0.05% | Cocoyl carboxymethydroxyethyl | 1% | 3 μm | 3.1 μm | Excellent |
| | | | ethylenediamine | | | | |
| Example 169 | Becromethasone propionate | 0.05% | Laurylaminopropionic acid | 1% | 3 μm | 3.1 μm | Excellent |
| Example 170 | Becromethasone propionate | 0.05% | Lauryldimethylaminoacetic acid betaine | 1% | 3 μm | 3.1 μm | Excellent |
| Example 171 | Becromethasone propionate | 0.05% | Lauryl betaine | 1% | 3 μm | 3.1 μm | Excellent |
| Example 172 | Becromethasone propionate | 0.05% | Cocoyl alanine | 1% | 3 μm | 3.1 μm | Excellent |
| Example 173 | Becromethasone propionate | 0.05% | Coconut oil fatty acid arginine salt | 1% | 3 μm | 3.1 μm | Excellent |
| Example 174 | Becromethasone propionate | 0.05% | Cocamidopropylhydroxysulfobetaine | 1% | 3 μm | 3.1 μm | Excellent |
| Example 175 | Becromethasone propionate | 0.05% | Cocamidopropyldimethylamine oxide | 1% | 3 μm | 3.1 μm | Excellent |
| Example 176 | Becromethasone propionate | 0.05% | Laurylhydroxusulfobetaine | 1% | 3 μm | 3.1 μm | Excellent |
| Example 177 | Becromethasone propionate | 0.05% | Lauryldimethylamine oxide | 1% | 3 μm | 3.1 μm | Excellent |
| Example 178 | Becromethasone propionate | 0.05% | Cocoylarginine ethylpyrroridone | 1% | 3 μm | 3.1 μm | Excellent |
| | | | carboxylate | | | | |
| Example 179 | Becromethasone propionate | 0.05% | Alkyloxyhydroxypropylarginine | 1% | 3 μm | 3.1 μm | Excellent |
| | | | hydrochloride | | | | |
| Example 180 | Becromethasone propionate | 0.05% | Pyrroridone carboxylate | 5% | 3 μm | 3.1 μm | Excellent |
| Example 181 | Becromethasone propionate | 0.05% | tween20 | 5% | 3 μm | 3.1 μm | Excellent |
| Example 182 | Becromethasone propionate | 0.05% | tween80 | 5% | 3 μm | 3.1 μm | Excellent |
| Example 183 | Isoproterenol hydrochloride | 0.50% | Benzalkonium chloride | 1% | 3 μm | 3 μm | Excellent |
| Example 184 | Isoproterenol hydrochloride | 0.50% | Lauroylsarcosine | 1% | 3 μm | 3 μm | Excellent |
| Example 185 | Isoproterenol hydrochloride | 0.50% | Benzethonium chloride | 1% | 3 μm | 3 μm | Excellent |
| Example 186 | Isoproterenol hydrochloride | 0.50% | Lauramide propyl betaine | 1% | 3 μm | 3 μm | Excellen |
| Example 187 | Isoproterenol hydrochloride | 0.50% | Cocoyl glutamate | 1% | 3 μm | 3 μm | Excellent |
| Example 188 | Isoproterenol hydrochloride | 0.50% | CHAPS | 1% | 3 μm | 3 μm | Excellent |
| Example 189 | Isoproterenol hydrochloride | 0.50% | Dodecylbenzenesulfonic acid | 1% | 3 μm | 3 μm | Excellent |
| Example 190 | Isoproterenol hydrochloride | 0.50% | SDS | 1% | 3 μm | 3 μm | Excellent |
| Example 191 | Isoproterenol hydrochloride | 0.50% | Cocamidopropyl betaine | 1% | 3 μm | 3 μm | Excellent |
| Example 192 | Isoproterenol hydrochloride | 0.50% | Alkylcarboxymethylhydroxyethyl | 1% | 3 μm | 3 μm | Excellent |
| | | | imidazolinium betaine | | | | |
| Example 193 | Isoproterenol hydrochloride | 0.50% | Cocoyl glycine | 1% | 3 μm | 3 μm | Excellent |
| Example 194 | Isoproterenol hydrochloride | 0.50% | carboxymethylhydroxyethyl | 1% | 3 μm | 3 μm | Excellent |
| | | | ethylenediamine | | | | |
| Example 195 | Isoproterenol hydrochloride | 0.50% | Laurylaminopropionic acid | 1% | 3 μm | 3 μm | Excellent |
| Example 196 | Isoproterenol hydrochloride | 0.50% | Lauryldimethylaminoacetic acid betaine | 1% | 3 μm | 3 μm | Excellent |
| Example 197 | Isoproterenol hydrochloride | 0.50% | Lauryl betaine | 1% | 3 μm | 3 μm | Excellent |
| Example 198 | Isoproterenol hydrochloride | 0.50% | Cocoyl alanine | 1% | 3 μm | 3 μm | Excellent |
| Example 199 | Isoproterenol hydrochloride | 0.50% | Coconut oil fatty acid arginine salt | 1% | 3 μm | 3 μm | Excellent |
| Example 200 | Isoproterenol hydrochloride | 0.50% | Cocamidoprpylhydroxysulfobetaine | 1% | 3 μm | 3 μm | Excellent |
| Example 201 | Isoproterenol hydrochloride | 0.50% | Cocamidopropyldimethylamine oxide | 1% | 3 μm | 3 μm | Excellent |
| Example 202 | Isoproterenol hydrochloride | 0.50% | Laurylhydroxysulfobetaine | 1% | 3 μm | 3 μm | Excellent |
| Example 203 | Isoproterenol hydrochloride | 0.50% | Lauryldimethylamine oxide | 1% | 3 μm | 3 μm | Excellent |
| Example 204 | Isoproterenol hydrochloride | 0.50% | Cocoylarginine ethylpyrroridone | 1% | 3 μm | 3 μm | Excellent |
| | | | carboxylate | | | | |
| Example 205 | Isoproterenol hydrochloride | 0.50% | Alkyloxyhydroxypropylarginine | 1% | 3 μm | 3 μm | Excellent |
| | | | hydrochloride | | | | |
| Example 206 | Isoproterenol hydrochloride | 0.50% | Pyrroridone carboxylate | 1% | 3 μm | 3 μm | Excellent |
| Example 207 | Isoproterenol hydrochloride | 0.50% | tween20 | 1% | 3 μm | 3 μm | Excellent |
| Example 208 | Isoproterenol hydrochloride | 0.50% | tween80 | 1% | 3 μm | 3 μm | Excellent |
| Example 209 | Procaterol hydrochloride | 0.01% | Benzalkonium chloride | 1% | 3 μm | 3 μm | Excellent |
| Example 210 | Procaterol hydrochloride | 0.01% | Lauroylsarcosine | 1% | 3 μm | 3 μm | Excellent |
| Example 211 | Procaterol hydrochloride | 0.01% | Benzethonium chloride | 1% | 3 μm | 3 μm | Excellent |
| Example 212 | Procaterol hydrochloride | 0.01% | Lauramide propyl betaine | 1% | 3 μm | 3 μm | Excellent |
| Example 213 | Procaterol hydrochloride | 0.01% | Cocoyl glutamate | 1% | 3 μm | 3 μm | Excellent |
| Example 214 | Procaterol hydrochloride | 0.01% | CHAPS | 1% | 3 μm | 3 μm | Excellent |
| Example 215 | Procaterol hydrochloride | 0.01% | Dodecylbenzenesulfonic acid | 1% | 3 μm | 3 μm | Excellent |
| Example 216 | Procaterol hydrochloride | 0.01% | SDS | 1% | 3 μm | 3 μm | Excellent |
| Example 217 | Procaterol hydrochloride | 0.01% | Cocamidopropyl betaine | 1% | 3 μm | 3 μm | Excellent |
| Example 218 | Procaterol hydrochloride | 0.01% | Alkylcarboxymethylhydroxyethyl | 1% | 3 μm | 3 μm | Excellent |
| | | | imidazolinium betaine | | | | |
| Example 219 | Procaterol hydrochloride | 0.01% | Cocoyl glycine | 1% | 3 μm | 3 μm | Excellent |
| Example 220 | Procaterol hydrochloride | 0.01% | Cocoyl carboxymethylhydroxyethyl | 1% | 3 μm | 3 μm | Excellent |
| | | | ethylenediamine | | | | |
| Example 221 | Procaterol hydrochloride | 0.01% | Laurylaminopropionic acid | 1% | 3 μm | 3 μm | Excellent |
| Example 222 | Procaterol hydrochloride | 0.01% | Lauryldimethylaminoacetic acid betaine | 1% | 3 μm | 3 μm | Excellent |
| Example 223 | Procaterol hydrochloride | 0.01% | Lauryl betaine | 1% | 3 μm | 3 μm | Excellent |
| Example 224 | Procaterol hydrochloride | 0.01% | Cocoyl alanine | 1% | 3 μm | 3 μm | Excellent |
| Example 225 | Procaterol hydrochloride | 0.01% | Coconut oil fatty acid arginine salt | 1% | 3 μm | 3 μm | Excellent |
| Example 226 | Procaterol hydrochloride | 0.01% | Cocamidopropylhydroxysulfobetaine | 1% | 3 μm | 3 μm | Excellent |
| Example 227 | Procaterol hydrochloride | 0.01% | Cocamidopropyldimethylamine oxide | 1% | 3 μm | 3 μm | Excellent |
| Example 228 | Procaterol hydrochloride | 0.01% | Laurylhydroxysulfobetaine | 1% | 3 μm | 3 μm | Excellent |
| Example 229 | Procaterol hydrochloride | 0.01% | Lauryldimethyllamine oxide | 1% | 3 μm | 3 μm | Excellent |
| Example 230 | Procaterol hydrochloride | 0.01% | Cocoylarginine ethylpyrroridone | 1% | 3 μm | 3 μm | Excellent |
| | | | carboxylate | | | | |
| Example 231 | Procaterol hydrochloride | 0.01% | Alkyloxyhydroxypropylarginine | 1% | 3 μm | 3 μm | Excellent |
| | | | hydrochloride | | | | |
| Example 232 | Procaterol hydrochloride | 0.01% | Pyrroidone carboxylate | 1% | 3 μm | 3 μm | Excellent |
| Example 233 | Procaterol hydrochloride | 0.01% | tween20 | 1% | 3 μm | 3 μm | Excellent |
| Example 234 | Procaterol hydrochloride | 0.01% | tween80 | 1% | 3 μm | 3 μm | Excellent |
| Comparative | None | | — | — | 3 μm | 3 μm | Good |
| Example 1 | | | | | | | |
| Comparative | Ipratropium bromide | 0.03% | — | — | 3 μm | — | Poor |
| Example 2 | | | | | | | |
| Comparative | Fenoterol hydrobromide | 0.10% | — | — | 3 μm | — | Poor |
| Example 3 | | | | | | | |
| Comparative | Sodium salbutamol sulfate | 1% | — | — | 3 μm | — | Poor |
| Example 4 | | | | | | | |
| Comparative | Sodium cromoglycate | 1% | — | — | 3 μm | — | Poor |
| Example 5 | | | | | | | |
| Comparative | Procaterol hydrochloride | 0.01% | — | — | 6 μm | — | Poor |
| Example 6 | | | | | | | |
| Comparative | Isoproterenol hydrochloride | 0.50% | — | — | 6 μm | — | Poor |
| Example 7 | | | | | | | |
| Comparative | Becromethasone propionate | 0.05% | — | — | 6 μm | — | Poor |
| Example 8 | | | | | | | |
| Comparatve | Fluticasone propionate | 0.05% | — | — | 6 μm | — | Poor |
| Example 9 | | | | | | | |
| Comparative | Acetylcysteine | 0.50% | — | — | 6 μm | — | Poor |
| Example 10 | | | | | | | |
| Comparative | Ipratropium bromide | 0.03% | Glycine betaine | — | 3 μm | — | Good |
| Example 11 | | | | | | | |
| Comparative | Sodium salbutamol sulfate | 1% | Glycine betaine | — | 3 μm | — | Good |
| Example 12 | | | | | | | |

As the drug ingredients, ipratropium bromide (0.03%) in Examples 1 to 26, fenoterol hydrobromide (0.15%) in Examples 27 to 52, sodium salbutamol sulfate (1.00%) in Examples 53 to 78, sodium cromoglycate (1.00%) in Examples 79 to 104, acetylcysteine (0.50%) in Examples 105 to 130, fluticasone propionate (0.05%) in Examples 131 to 156, becromethasone propionate (0.05%) in Examples 157 to 182, isoproterenol hydrochloride (0.50%) in Examples 183 to 208, and procaterol hydrochloride (0.01%) in Examples 209 to 234 were used.

As the surface-active compounds, all the following. 26 types of compounds were used in each Example group:

Benzalkonium chloride; Lauroylsarcosine; Benzethonium chloride; Lauramide propyl betaine; Cocoyl glutamate; CHAPS; Dodecylbenzenesulfonic acid; SDS; Cocamidopropyl betaine; Alkylcarboxymethylhydroxyethyl imidazolinium betaine; Cocoyl glycine; Cocoyl carboxymethylhydroxyethyl ethylenediamine; Laurylaminopropionic acid; Lauryldimethylaminoacetic acid betaine; Lauryl betaine; Cocoyl alanine; Coconut oil fatty acid arginine salt; Cocamidopropylhydroxysulfobetaine; Cocamidopropyldimethylamine oxide; Laurylhydroxysulfobetaine; Lauryldimethylamine oxide; Cocoylarginine ethylpyrroridone carboxylate; Alkyloxyhydroxypropylarginine hydrochloride; Pyrroridone carboxylate; tween20; and tween80.

The contents of pyrroridone carboxylate, tween20 and tween80 were each 5% in Examples 24 to 26, 50 to 52, 76 to 78, 102 to 104, 128 to 130, 154 and 156, and 180 to 182 and were each 1% in Examples 206 to 208 and 232 to 234. The contents of other compounds were 1%.

In Comparative Examples, since surface-active compounds were not contained, ejection was not or hardly achieved without any correlation to the type of drugs and the presence of additives. When glycine betaine was added as shown in Comparative Examples 11 and 12, the ejection was achieved to some degree, but a sufficient stability was not observed. On the other hand, in Examples, the ejection was normally achieved and was stable. The results of HPLC analysis confirmed that the peak position, peak area, and liquid composition were not changed in Examples before and after the ejection.

### Examples 235 to 244

(Ejection of Plural Drug Compounds)

Ejection liquids each containing a plurality of drug compounds were prepared. These ejection liquids were evaluated by the same manner in Example 1. Table 2 shows the prescription investigated in these Examples and results thereof. The results of HPLC analysis of these Examples show that no change in the peak chart and in the liquid composition was confirmed before and after the ejection.

TABLE 2

| | | | | | | | Ejection |
|---|---|---|---|---|---|---|---|
| | Drug Compound | | Surface Active Compound | | Nozzle | Particle | Character- |
| | Type | Concentration | Type | Concentration | Diameter | Size | istic |
| Example 235 | Ipratropium bromide | 0.03% | Benzalkonium chloride | 1% | 3 μm | 3 μm | Excellent |
| | Fenoterol hydrobromide | 0.15% | | | | | |
| Example 236 | Ipratropium bromide | 0.03% | Lauroylsarcosine | 1% | 3 μm | 3 μm | Excellent |
| | Fenoterol hydrobromide | 0.15% | | | | | |
| Example 237 | Ipratropium bromide | 0.30% | Benzalkonium chloride | 5% | 3 μm | 3 μm | Excellent |
| | Fenoterol hydrobromide | 1.50% | | | | | |
| Example 238 | Ipratropium bromide | 0.30% | Lauroylsarcosine | 5% | 3 μm | 3 μm | Excellent |
| | Fenoterol hydrobromide | 1.50% | | | | | |
| Example 239 | Ipratropium bromide | 1.00% | Benzalkonium chloride | 5% | 3 μm | 3 μm | Excellent |
| | Fenoterol hydrobromide | 4.00% | | | | | |
| Example 240 | Sodium cromoglycate | 1.00% | Benzalkonium chloride | 1% | 3 μm | 3 μm | Excellent |
| | Fenoterol hydrobromide | 0.15% | | | | | |
| Example 241 | Sodium cromoglycate | 1.00% | Lauroylsarcosine | 1% | 3 μm | 3 μm | Excellent |
| | Fenoterol hydrobromide | 0.15% | | | | | |
| Example 242 | Sodium cromoglycate | 5.00% | Benzalkonium chloride | 5% | 3 μm | 3 μm | Excellent |
| | Fenoterol hydrobromide | 1.50% | | | | | |
| Example 243 | Sodium cromoglycate | 5.00% | Lauroylsarcosine | 5% | 3 μm | 3 μm | Excellent |
| | Fenoterol hydrobromide | 1.50% | | | | | |
| Example 244 | Sodium cromoglycate | 5.00% | Benzalkonium chloride | 5% | 3 μm | 3 μm | Excellent |
| | Fenoterol hydrobromide | 4.00% | | | | | |

It was confirmed that a solution containing a plurality of drug compounds could be also ejected.

### Examples 245 to 250 and Comparative Examples 13 to 20

(Synergetic Effect Due to Surface-Active Compound and Alcohol and Buffer

Ejection liquids were each prepared by adding ethanol to a solution containing a drug compound and a surface-active compound. In addition, ejection liquids were similarly prepared using a buffer. These ejection liquids were evaluated by ejection test as in Example 1. Table 3 shows the prescription investigated in these Examples and results thereof.

TABLE 3

| | | | | | Alcohol/Buffer | | | | Ejection |
|---|---|---|---|---|---|---|---|---|---|
| | Drug Compound | | Surface Active Compound | | | Concen- | Nozzle | Particle | Character- |
| | Type | Concentration | Type | Concentration | Type | tration | Diameter | Size | istic |
| Example 245 | Ipratropium bromide | 1.00% | Benzalkonium chloride | 1% | Ethanol | 25% | 3 μm | 3 μm | Excellent |
| | Fenoterol hydrobromide | 4.00% | | | | | | | |
| Example 246 | Sodium cromoglycate | 5.00% | Benzalkonium chloride | 1% | Ethanol | 25% | 3 μm | 3 μm | Excellent |
| | Fenoterol hydrobromide | 4.00% | | | | | | | |
| Example 247 | Ipratropium bromide | 1.00% | Benzalkonium chloride | 4% | Ethanol | 25% | 3 μm | 3 μm | Excellent |
| | Fenoterol hydrobromide | 4.00% | | | EDTA buffer | | | | |
| Example 248 | Sodium cromoglycate | 5.00% | Benzalkonium chloride | 4% | Ethanol | 25% | 3 μm | 3 μm | Excellent |
| | Fenoterol hydrobromide | 4.00% | | | EDTA buffer | | | | |
| Example 249 | Ipratropium bromide | 1.00% | Lauroylsarcosine | 6% | Ethanol | 25% | 3 μm | 3 μm | Excellent |
| | Fenoterol hydrobromide | 4.00% | | | EDTA buffer | | | | |
| Example 250 | Sodium cromoglycate | 5.00% | Lauroylsarcosine | 4% | Ethanol | 25% | 3 μm | 3 μm | Excellent |
| | Fenoterol hydrobromide | 4.00% | | | Citrate buffer | | | | |
| Comparative | Ipratropium bromide | 1.00% | Benzalkonium chloride | 1% | | | 3 μm | 3 μm | Good |
| Example 13 | Fenoterol hydrobromide | 4.00% | | | | | | | |
| Comparative | Sodium cromoglycate | 5.00% | Benzalkonium chloride | 1% | — | — | 3 μm | 3 μm | Good |
| Example 14 | Fenoterol hydrobromide | 4.00% | | | | | | | |
| Comparative | — | — | — | — | Ethanol | 25% | 3 μm | — | Good |
| Example 15 | | | | | | | | | |
| Comparative | Ipratropium bromide | 0.03% | — | — | Ethanol | 25% | 3 μm | — | Poor |
| Example 16 | Fenoterol hydrobromide | 0.15% | | | | | | | |
| Comparative | Sodium cromoglycate | 1.00% | — | — | Ethanol | 25% | 3 μm | — | Poor |
| Example 17 | Fenoterol hydrobromide | 0.15% | | | | | | | |

| | | | | | | | | |
|---|---|---|---|---|---|---|---|---|
| Comparative | — | — | — | — | EDTA buffer | 3 μm | — | Good |
| Example 18 | | | | | | | | |
| Comparative | Ipratropium bromide | 0.03% | — | — | EDTA buffer | 3 μm | — | Poor |
| Example 19 | Fenoterol hydrobromide | 0.15% | | | | | | |
| Comparative | Sodium cromoglycate | 1.00% | — | — | EDTA buffer | 3 μm | — | Poor |
| Example 20 | Fenoterol hydromide | 0.15% | | | | | | |

It was confirmed that an ejection liquid could be ejected even if the amount of a surface-active compound was low by adding alcohol and/or buffer in addition to the surface-active compound to the ejection liquid. Further, even if the amount of a surface-active compound in an ejection liquid was too small to ejection the ejection liquid with the surface-active compound alone, the ejection liquid could be ejected by adding the alcohol and/or buffer. As shown by Comparative Examples, the ejection liquids prepared with alcohol or buffer only could not be ejected. The results of HPLC analysis of these Examples show that no change in the peak chart and in the liquid composition was confirmed before and after the ejection.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-083399, filed Mar. 24, 2006, which is hereby incorporated by reference herein in its entirety.

What is claimed is:

1. An ejection liquid being ejected from an ejection opening by an ink jet system and being used for treating respiratory disease, the ejection liquid comprising:
an active ingredient for treating respiratory disease;
a surface-active compound represented by the following Formula (1):
R−A (1)
wherein R denotes a substituted or unsubstituted, saturated or unsaturated aliphatic chain of 3 or more and 30 or less carbon atoms or a saturated or unsaturated aliphatic derivative of 3 or more and 30 or less carbon atoms, and
A comprises at least one selected from the group consisting of a substituted or unsubstituted carboxylic acid group, a substituted or unsubstituted sulfonic acid group, a substituted or unsubstituted anion and its counter ion, a substituted or unsubstituted quaternary ammonium group, a substituted or unsubstituted cation and its counter ion, and a betaine skeleton,
or an amine oxide or water-soluble nonionic compound having a substituted or unsubstituted, saturated or unsaturated aliphatic chain of 3 or more and 30 or less carbon atoms; and
a liquid medium.

2. The ejection liquid according to claim 1, wherein the surface-active compound represented by Formula (1) comprises a substituted or unsubstituted alkyl group of 3 or more and 30 or less carbon atoms and is at least one selected from the group consisting of:
alkyl amino acid,
alkyl betaine,
fatty acyl betaint,
fatty acyl alkyl betaine,
alkyl imidazolinium betaine,
fatty acyl amino acid,
alkyl sulfonic acid,
alkylbenzene sulfonic acid,
alkylether amino acid salt,
acylamino acid pyrroridone carboxylate,
alkylamine oxide,
fatty acyl amine oxide,
fatty amino acid salt,
ethylene diamine derivative,
pyrroridone carboxylic acid,
benzalkonium salt,
benzethonium salt, and
fatty acyl polyoxyethylene sorbitan alkylate.

3. The ejection liquid according to claim 1, wherein the active ingredient for treating respiratory disease is at least one selected from the group consisting of cromoglycate, salbutamol, ipratropium, fenoterol, isoproterenol, trimetoquinol, procaterol, salmeterol, oxitropium, beclomethasone propionate, bromhexine, acetylcysteine, budesonide, fluticasone propionate, derivatives of these compounds, salts of these compounds and their derivatives, and a mixture of at least two of these compounds and their derivatives.

4. The ejection liquid according to claim 1, wherein the ejection liquid is ejected from an ejection opening of an ejection device by thermal energy.

5. A method for ejecting a liquid, wherein an ejection liquid according to claim 1 is ejected by an ink jet system from a device ejecting a drug for treating respiratory disease.

6. The method for ejecting a liquid according to claim 5, wherein the ink jet system is a thermal ink jet system using thermal energy.

7. A liquid ejection cartridge, the cartridge comprising a tank for storing an ejection liquid according to claim 1 and an ejection head.

8. The liquid ejection cartridge according to claim 7, wherein the ejection head ejects a liquid by a thermal ink jet system.

9. An ejection device for treating respiratory disease, the ejection device comprising the cartridge according to claim 7; and a channel and an opening for leading a liquid to be ejected from a liquid-ejecting portion of a head of the cartridge to an inhalation region of a user.

10. The ejection device according to claim 9, the device being used for inhalation through a mouth of a user.
